SEQUENCE LISTING (30 sequences)

SEQ ID NO: 1 - DNA, 450 bp, Homo sapiens
cttcttcagcctgtccggcctcagcatcatctgcgccatgagtgtcgagcgctacctggccatcaaccatgcctatttctacagccactacgtggacaagcgattggcgggcctcacgctctttgcagtctatgcgtccaacgtgctcttttgcgcgctgcccaacatgggtctcggtagctcgcggctgcagtacccagacacctggtgcttcatcgactggaccaccaacgtgacggcgcacgccgcctactcctacatgtacgcgggcttcagctccttcctcattctcgccaccgtcctctgcaacgtgcttgtgtgcggcgcgctgctccgcatgcaccgccagttcatgcgccgcacctcgctgggcaccgagcagcaccacgcggccgcggccgcctcggttgcctcccggggccaccccgctgcctccccagccttgccgcg

SEQ ID NO: 2 - DNA, 443 bp, Homo sapiens
ctgcgagagcgcgcccagccccgccttcgggccccacagtccctgcacccaggtttccattgcgcggctctcctcagctccttcccgccgcccagtctggatcctgggggaggcgctgaagtcggggcccgccctgtggccccgcccggcccgcgcttgctagcgcccaaagccagcgaagcacgggcccaaccgggccatgtcgggggagcctgagctcattgagctgcgggagctggcacccgctgggcgcgctgggaagggccgcacccggctggagcgtgccaacgcgctgcgcatcgcgcggggcaccgcgtgcaaccccacacggcagctggtccctggccgtggccaccgcttccagcccgcggggcccgccacgcacacgtggtgcgacctctgtggcgacttcatctggggcgtcgtgcgcaaaggcctgcagt

SEQ ID NO: 3 - DNA, 490 bp, Homo sapiens
tggctctctgcctaccgcaaacttgctggtctaatttaggaacaattgggccgaaaggtatcagcgagagcaacagaccccggtgttgtgccgcacagggagccgcatccgcagacgcccctcgctgcccctgggctcgggccaaaccctgcataaggtcccctggacagccaggtaatctccgtcccgcctgcccgaccggggtcgcacgagcacaggcgcccacgccatgttggctgcccaaagggctcgccgcccaagccgggccagaaggcaggaggcggaaaaccagcctccggtggcgggcgaaagcaaccgctctttctgttctctcttcgccctccctcgtggaaacgcagactcgaccctaaacgcttaacccacagagatcaacaggttcaagcggaatattcgcgatcctcggtttctattggttgctcaaagccttttcatgcaaccagcagctcggatgtttaataaaatatgaatt

SEQ ID NO: 4 - DNA, 25 bp, Artificial Sequence
  primer
ttagatatttggtgttttatcgatt

SEQ ID NO: 5 - DNA, 22 bp, Artificial Sequence
  primer
aaaaactaaaacccgcgtacat

SEQ ID NO: 6 - DNA, 38 bp, Artificial Sequence
  blocking primer
ttttattgattggattattaatgtgatggtgtatgttg

SEQ ID NO: 7 - DNA, 28 bp, Artificial Sequence
  probe
ataaacgacgtacgccgtcacgttaata

SEQ ID NO: 8 - DNA, 24 bp, Artificial Sequence
  primer
tgggtattgtagtcgcgagttatc

SEQ ID NO: 9 - DNA, 22 bp, Artificial Sequence
  primer
ctacgtaaacaaacgattaacg

SEQ ID NO: 10 - DNA, 32 bp, Artificial Sequence
  blocking primer
tgtgagttattgagatttatgttgggtagtgt

SEQ ID NO: 11 - DNA, 35 bp, Artificial Sequence
  probe
caatctatacgtccaacgtactcttttacgcgcta

SEQ ID NO: 12 - DNA, 19 bp, Artificial Sequence
  primer
gcgttgaagtcggggttcg

SEQ ID NO: 13 - DNA, 19 bp, Artificial Sequence
  primer
ccgattaaacccgtacttc

SEQ ID NO: 14 - DNA, 33 bp, Artificial Sequence
  blocking primer
ttggggtttgttttgtggtttcgtttggtttgt

SEQ ID NO: 15 - DNA, 20 bp, Artificial Sequence
  probe
cgctaacaaacgcgaaccga

SEQ ID NO: 16 - DNA, 19 bp, Artificial Sequence
  primer
gggagtttgagtttattga

SEQ ID NO: 17 - DNA, 21 bp, Artificial Sequence
  primer
gatacgcaacgcgttaacacg

SEQ ID NO: 18 - DNA, 33 bp, Artificial Sequence
  blocking primer
cacattaacacactccaaccaaatacaaccctt

SEQ ID NO: 19 - DNA, 21 bp, Artificial Sequence
  probe
cgcccaacgaataccaactcc

SEQ ID NO: 20 - DNA, 18 bp, Artificial Sequence
  primer
gttcgtgcgatttcggtc

SEQ ID NO: 21 - DNA, 19 bp, Artificial Sequence
  primer
tcgctacccctaaactcga

SEQ ID NO: 22 - DNA, 25 bp, Artificial Sequence
  blocking primer
tgattttggttgggtaggtgggatg

SEQ ID NO: 23 - DNA, 23 bp, Artificial Sequence
  probe
caaccaaataatctccgtcccgc

SEQ ID NO: 24 - DNA, 17 bp, Artificial Sequence
  primer
ggcgggcgaaagtaatc

SEQ ID NO: 25 - DNA, 20 bp, Artificial Sequence
  primer
cgaaaatcgcgaatattccg

SEQ ID NO: 26 - DNA, 43 bp, Artificial Sequence
  blocking primer
acaaatattccacttaaacctattaatctctataaattaaaca

SEQ ID NO: 27 - DNA, 27 bp, Artificial Sequence
  probe
aaaatcgaatctacgtttccacgaaaa

SEQ ID NO: 28 - DNA, 23 bp, Artificial Sequence
  primer
gtgatggaggaggtttagtaagt

SEQ ID NO: 29 - DNA, 23 bp, Artificial Sequence
  primer
ccaataaaacctactcctccctt

SEQ ID NO: 30 - DNA, 30 bp, Artificial Sequence
  probe
accaccacccaacacacaataacaaacaca

(19) United States
(12) Patent Application Publication
(10) Pub. No.: US 2024/0240260 A1
(43) Pub. Date: Jul. 18, 2024

(54) METHOD OF IDENTIFYING LUNG CANCER WITH METHYLATION BIOMARKER GENES AND RADIOLOGICAL CHARACTERISTIC

(71) Applicant: Excellen Medical Thchnology Co., Ltd, Changping District Beijing (CN)

(72) Inventors: Mingming Li, Changping District Beijing (CN); Jue Pu, Changping District Beijing (CN)

(21) Appl. No.: 18/561,984

(22) PCT Filed: May 17, 2021
(86) PCT No.: PCT/CN2021/094016

§ 371 Date: Nov. 17, 2023

(51) Int. Cl. C12Q 1/6886; C12Q 1/6851; G16B 20/20; G16B 40/20

(52) U.S. Cl. CPC C12Q 1/6886; C12Q 1/6851; G16B 20/20; G16B 40/20; C12Q 2600/154

(57) ABSTRACT

A method for identifying lung cancer in a subject with a pulmonary nodule, comprising: determining the diameter of the pulmonary nodule the subject; detecting methylation levels of methylation biomarker genes PTGER4, RASSF1A and SHOX2 in a sample from the subject; and assessing whether the subject has a lung cancer or not by using the diameter in combination with the detected methylation levels. The method is able to distinguish between malignant and benign lung nodules.

### CROSS-REFERENCE TO RELATED APPLICATIONS

This application is a national phase entry under 35 U.S.C. § 371 of International Patent Application PCT/CN2021/094016, filed May 17, 2021, designating the United States of America and published as International Patent Publication WO 2022/241599 A1 on Nov. 24, 2022.

### TECHNICAL FIELD

The present disclosure relates to a method of identifying lung cancer in a subject with a pulmonary nodule. More specifically, the present disclosure relates to a lung cancer diagnosis method by using multiple methylation biomarker genes in combination with a radiological characteristic of a pulmonary nodule.

### BACKGROUND

Lung cancer is the second most common cancer globally and the leading cause of cancer mortality worldwide [1]. In 1987, it surpassed breast cancer as the leading cause of cancer-related deaths of women. By 2020, Lung cancer is expected to account for 22% of all female cancer deaths and 23% of all male cancer deaths [1].

The exceptional high mortality of lung cancer can be attributed to a high degree by late diagnosis. The 5-year survival rate of lung cancer is only 15-19% at all stages. Outcomes can be significantly better at an early-stage diagnosis, especially for stage I, the 5-year survival rate can increase up to 81-85% [2, 3]. Thus, it seems reasonable to improve lung cancer screening at earlier stages. Low-dose computed tomography (LDCT) is widely accepted as a reliable screening tool for lung cancer early detection. The National Lung Screening Trial (NLST) reported that LDCT decreases the mortality rate by 20% in high-risk people [4, 5]. However, pulmonary nodules (PNs) are encountered with increased frequency in asymptomatic individuals due to the widespread use of LDCT. High false-positive rates and overdiagnosis limited the diagnostic accuracy of LDCT screening. The National Lung Screening Trial showed that in heavy smokers, the positive rate of indeterminate PNs detected by LDCT was 24.2%; however, 96.4% of these PNs were ultimately confirmed to be false positives over the three rounds of screening [5].

Currently, to predict the malignancy probability of PNs found by LDCT, a series of examination techniques have been proposed, including non-invasive and invasive approaches [6]. Each approach has advantages and disadvantages. Noninvasive approaches include follow-up with positron emission tomography, LDCT, or magnetic resonance imaging for up to 2 years to determine whether it is a benign lesion. These non-invasive approaches often result in unnecessary radiation exposure, anxiety, procedures, and additional cost for subjects with benign lesions. A CT-guided transthoracic needle biopsy can establish a specific benign or malignant diagnosis but is invasive, potentially risky, and sometimes non-diagnostic [7]. Thus, it is clinically significant to develop new approaches to accurately identify patients with malignant from benign PNs safely and cost-effectively.

Analysis of lung tumor-associated molecular changes in body fluids may provide a safe and cost-effective approach for detecting lung cancer. DNA methylation is a relatively stable biochemical modification; it can be detected not only from tissue but also in serum and plasma [8]. Assessment of DNA methylation in plasma offers a potentially cost-effective method in discriminating malignant from benign PNs. Prostaglandin E receptor 4 gene (PTGER4), ras association domain family 1A (RASSF1A), and short stature homeobox gene two (SHOX2) methylation have been separately identified as valuable biomarkers for lung cancer diagnosis in several research studies [9,10,11,12]. However, investigating whether the three methylation biomarkers are useful in distinguishing lung cancer among individuals with LDCT-detected PNs has hardly been reported.

Previous studies also showed that, based on subjects' demographic characteristics and radiological features of PNs on CT images, the constructed predictive models could identify malignant from benign PNs [13,14,15,16]. For example, Swensen et al. developed a Mayo Clinic model based on six independent predictors (patients' age, smoking history, cancer history, nodule diameter, upper lobe position, and spiculation), which had an AUC of 0.83 for the diagnosis of malignant PNs [13]. Gould et al. established another prediction model, which yielded 0.78 AUC based on age, smoking history, nodule diameter, and smoking cessation [14, 15]. Recently, McWilliams et al. also developed two similar prediction models, with AUCs of 0.89-0.91 [16]. Although these clinical/radiological characteristics-based models are promising in identifying malignant PNs, the diagnostic accuracy still need improvement.

Considering the complex tumor microenvironment and clonal selection in lung cancer development, using circulating biomarkers alone or clinical/radiological factors alone might not have sufficient diagnostic accuracy for lung cancer.

### BRIEF SUMMARY

In one aspect, the present disclosure provides use of multiple methylation biomarker genes in combination with a radiological characteristic of a pulmonary nodule for the prediction of lung cancer in a subject.

In some embodiments, the methylation biomarker genes are selected from PTGER4, RASSF1A and SHOX2.

In some embodiments, the multiple methylation biomarker genes are PTGER4, RASSF1A and SHOX2.

In some embodiments, the radiological characteristic is the size of the pulmonary nodule.

In some embodiments, the radiological characteristic is the diameter of the pulmonary nodule.

In some embodiments, the subject has multiple pulmonary nodules, and the radiological characteristic is the diameter of the largest pulmonary nodule.

In some embodiments, the diameter is a CT-derived diameter.

In some embodiments, the diameter is a LDCT-derived diameter.

In some embodiments, the lung cancer is a malignant pulmonary nodule.

In another aspect, the present disclosure provides a method for identifying lung cancer in a subject with a pulmonary nodule, comprising: determining a radiological characteristic of the pulmonary nodule the subject; detecting methylation levels of multiple methylation biomarker genes in a sample from the subject; and assessing whether the subject has a lung cancer or not by using the radiological characteristic in combination with the detected methylation levels.

In some embodiments, the methylation biomarker genes are selected from PTGER4, RASSF1A and SHOX2.

In some embodiments, the multiple methylation biomarker genes are PTGER4, RASSF1A and SHOX2.

In some embodiments, detection of methylation levels is performed by using a methylation-specific primer pair.

In some embodiments, the methylation-specific primer pair for PTGER4 gene comprises the sequences of SEQ ID NOs:4 and 5 or the sequences of SEQ ID NOs:8 and 9; the methylation-specific primer pair for RASSF1A gene comprises the sequences of SEQ ID NOs:12 and 13 or the sequences of SEQ ID NOs:16 and 17; the methylation-specific-primer pair for SHOX2 gene comprises the sequences of SEQ ID NOs:20 and 21 or the sequences of SEQ ID NOs:24 and 25.

In some embodiments, the radiological characteristic is the size of the pulmonary nodule.

In some embodiments, the radiological characteristic is the diameter of the pulmonary nodule.

In some embodiments, the subject has multiple pulmonary nodules, and the radiological characteristic is the diameter of the largest pulmonary nodule.

In some embodiments, the diameter is a CT-derived diameter.

In some embodiments, the diameter is a LDCT-derived diameter.

In some embodiments, the assessing is based on a prediction model constructed by using a training cohort consisting of patients with a malignant pulmonary nodule and patients with a benign pulmonary nodule, and the information about the pulmonary nodule radiological characteristic and levels of methylation biomarker genes as well as whether the subject is a lung cancer patient is known in the training cohort.

In some embodiments, the prediction model is a logistic regression model.

In some embodiments, the lung cancer is a malignant pulmonary nodule.

In some embodiments, the prediction model is presented as:

the probability of malignant PN=e^{x}/(1+e^{x}), where e is the base of the natural logarithm and x=−4.433+1.066×(8.327−0.103*2^{-ΔCT.SHOX2}−0.184*2^{-ΔCT.RASSF1A}−0.077*2^{-ΔCT.PTGER4)}+0.151×the diameter of the pulmonary nodule.

In another aspect, the present disclosure provides a kit for identifying lung cancer in a subject with a pulmonary nodule, comprising agents for detecting methylation levels of multiple methylation biomarker genes PTGER4, RASSF1A and SHOX2 in a sample from the subject.

In some embodiments, the agents for detecting methylation levels comprise methylation-specific primer pairs, and wherein the methylation-specific primer pair for PTGER4 gene comprises the sequences of SEQ ID NOs:4 and 5 or the sequences of SEQ ID NOs:8 and 9; the methylation-specific primer pair for RASSF1A gene comprises the sequences of SEQ ID NOs:12 and 13 or the sequences of SEQ ID NOs:16 and 17; and the methylation-specific primer pair for SHOX2 gene comprises the sequences of SEQ ID NOs:20 and 21 or the sequences of SEQ ID NOs:24 and 25.

In some embodiments, the kit further comprises an instruction indicating that the methylation levels are used in combination with a radiological characteristic of the pulmonary nodule to identify whether the subject is a lung cancer patient.

In some embodiments, the radiological characteristic is the diameter of the pulmonary nodule.

In some embodiments, the lung cancer is a malignant pulmonary nodule.

The methylation levels of three genes PTGER4, RASSF1A and SHOX2 in combination with a radiological characteristic (lung nodule diameter) are able to distinguish between malignant and benign lung nodules with an AUC of, e.g., 0.951.

### BRIEF DESCRIPTION OF THE DRAWINGS

FIG. 1 Comparison of the studied DNA methylation expressions in patients with benign PNs, and patients with malignant PNs in a training cohort. Scatter plots show the distribution of relative normalized methylation values for each of the 3 genes determined by q-PCR. The paired t-test was performed.

FIG. 2 Receiver-operator characteristic (ROC) curve analysis of the three models in a training cohort. The area under the ROC curve (AUC) for each model conveys its accuracy for diagnosing malignant PNs. The prediction model produced a higher AUC value for identifying malignant PNs comparing with the panel of the three DNA methylation biomarkers and the Mayo Clinic model.

FIG. 3 Comparison of the studied DNA methylation expressions in patients with benign PNs, and patients with malignant PNs in an independent cohort. Scatter plots show the distribution of relative normalized methylation values for each of the 3 genes determined by q-PCR. The paired t-test was performed.

FIG. 4 Comparison of ROC curves generated using the prediction model, panel of the three DNA methylation biomarkers, and Mayo Clinic model in an independent cohort. The prediction model produced the highest AUC value of the three models.

### DETAILED DESCRIPTION

Unless defined otherwise, technical and scientific terms used herein have the same meaning as commonly understood by a person of ordinary skill in the art. Any methods, devices and materials similar or equivalent to those described herein can be used in the practice of the present disclosure. The following definitions are provided to facilitate understanding of certain terms used herein and are not meant to limit the scope of the present disclosure.

The articles “a” and “an” are used herein to refer to one or to more than one (i.e., to at least one) of the grammatical object of the article. By way of example, “an element” means one element or more than one element.

“Radiological characteristic” used herein refers to a feature related to a pulmonary nodule, including, for example, the location, type, or size (e.g., diameter) of the pulmonary nodule. Such features can be acquired by using CT (computed tomography), especially Low-dose computed tomography (LDCT).

“Methylation biomarker gene” used herein refers to a gene, the methylation of which is associated with a particular disease state, such as a cancer. A methylation biomarker gene may also indicate a change in expression or state of a protein that correlates with the risk or progression of a disease, or with the susceptibility of the disease to a given treatment. A good methylation biomarker gene can be used to diagnose disease risk, presence of disease in an individual, or to tailor treatments for the disease in an individual. Methylation generally affects a cytosine in front of a guanine (CpG) on a DNA strand and the methylation in a promoter region of gene is of great importance to the function of the gene, such as the up-regulation or down-regulation its expression.

“Methylation level” used herein is an expression of the amount of methylation in one or more copies of a gene or nucleic acid sequence of interest. The methylation level may be calculated as an absolute measure of methylation within the gene or nucleic acid sequence of interest. A “methylation level” may also be determined as the amount of methylated DNA, relative to the total amount DNA present or as the number of methylated copies of a gene or nucleic acid sequence of interest, relative to the total number of copies of the gene or nucleic acid sequence. Additionally, the “methylation level” can be determined as the percentage of methylated CpG sites within the DNA region of interest. In one embodiment, the methylation level of the gene of interest is 15% to 100%, such as 50% to 100%, 60% to 100%, 70-100%, 80% to 100%, or 90% to 100%. Thus, in one embodiment of the present disclosure the methylation level of the genes according to the disclosure is 80%, 81%, 82%, 83%, 84%, 85%, 86%, 87%, 88%, 89%, 90%, 91%, 92%, 93%, 94%, 95%, 96%, 97%, 98%, 99% or 100%. Methylation-specific reagents for detecting methylation level of a gene, which can change the nucleotide sequence of a nucleic acid molecule in a manner that reflects the methylation state of the nucleic acid molecule, are known in the art. Methods of treating a nucleic acid molecule with such a reagent can include contacting the nucleic acid molecule with the reagent, coupled with additional steps, if desired, to accomplish the desired change of nucleotide sequence. Such methods can be applied in a manner in which unmethylated nucleotides (e.g., each unmethylated cytosine) is modified to a different nucleotide. For example, in some embodiments, such a reagent can deaminate unmethylated cytosine nucleotides to produce deoxy uracil residues. Examples of such reagents include, but are not limited to, a methylation-sensitive restriction enzyme, a methylation-dependent restriction enzyme, and a bisulfite reagent. For more information about the detection of methylation level of a gene with a bisulfite reagent, see, e.g., WO2019144275, the disclosure of which is incorporated herein by reference. For example, the detection of the methylation level may comprise extracting DNA from a biological sample, treating it with bisulfite, and then carrying out a PCR amplification by using a methylation-specific primer pair. The bisulfite treatment causes unmethylated cytosine residues in a double-stranded DNA molecule to deaminate to be uracils; while methylated cytosine residues remain unchanged. As a result, in the subsequent PCR amplification reaction, methylated cytosine residue sites on a template are paired with guanine residues in a primer as cytosine residues, while unmethylated cytosine residue sites are paired with adenine residues in a primer as uracil residues. When a target region of a biomarker gene is not methylated, the primer pair used cannot effectively pair with and bind to the target region (after treated with bisulfite) which is used as a template in the PCR amplification reaction, and cannot (or rarely) generate amplification products; and when the target gene of the biomarker gene is methylated, the primer pair used is able to effectively pair with and bind to the target region (after treated with bisulfite) which is used as a template in the PCR amplification reaction, and thus generate amplification products. The differences of these amplification reactions can be monitored in real time during the amplification reactions, or can be judged by detecting the amplification products.

“Logistic regression model”, also known as “logistic regression” or “logit model”, relates to a regression model where the dependent variable is categorical. Logistic regression measures the relationship between the categorical dependent variable and one or more independent variables by estimating probabilities using a logistic function. The method of creating of a logistic regression model by utilizing data from subjects with and without a disease of interest is well known in the art. Generally, the subjects can be divided randomly into a training cohort and a test cohort. The training cohort is used for training thereby creating the logistic regression model and the prediction, i.e., verification, is implemented by using the test cohort. The aforementioned courses are executed repeatedly for different subject division to optimize the coefficients of the logistic regression equation. The performance of the prediction may be confirmed by integrating the result of the prediction, for example, through receiver operating characteristic curves, i.e., ROC curves. The ROC curve is a plot of the true positive rate against the false positive rate for the different possible cut points of a diagnostic test. It shows the trade-off between sensitivity and specificity depending on the selected cut point (any increase in sensitivity will be accompanied by a decrease in specificity). As the performance of a logistic regression model becomes better, the area under the curve (AUC) increases. The area under an ROC curve (AUC) is a measure for the accuracy of a diagnostic test (the larger the area the better; the optimum is 1; and a random test would have a ROC curve lying on the diagonal with an area of 0.5).

“Subject” as used herein refers to an individual (preferably a human) suffering from or suspected of having a certain disease, or, when predicting the susceptibility, “subject” may also include healthy individuals. The term is generally used interchangeably with ““patient,” “test “subject,” “treatment” subject,” and the like.

In this study, one aim was to investigate if combining DNA methylation biomarkers with clinical/radiological characteristics could more efficiently distinguish malignant from benign lung nodules detected by LDCT. As a result, it was discovered that the methylation levels of three genes PTGER4, RASSF1A and SHOX2, in combination with a radiological characteristic (lung nodule diameter), were able to distinguish between malignant and benign lung nodules.

### Example

### Materials and Methods

### Ethics

Participants in Henan Cancer Hospital, the Affiliated Cancer Hospital of Zhengzhou University were enrolled. All participants signed the informed consent before blood collection, and they were informed of the usage of plasma and the test results. The current study has received approval from the Medical Ethics Committee of Henan Cancer Hospital (2018157).

### Patient Cohorts and Study Design

The participants were enrolled with lung nodules newly detected in Henan Cancer Hospital from January 2019 to December 2019. Blood samples from all subjects who met the selection criteria were obtained. The inclusion criteria were: (I) subjects detected pulmonary nodules on CT scans. (II) LDCT-derived nodule diameter between 4 and 35 mm; (III) the participants' clinical information should be complete. The exclusion criteria were: (I) pregnancy or lactation; (II) current pulmonary infection; (III) surgery within 6 months; (IV) radiotherapy within 1 year; and (V) life expectancy of <1 year.

For the selected patients, CT examinations were performed at the institution with the Revolution CT (General Electric Medical Systems, Milwaukee, Wisconsin, USA) or the Brilliance iCT (Philips Healthcare, Best, The Netherlands) using a tube voltage of 120 kV and a current of 200 mA. The target lesion was reconstructed with the following standard reconstruction parameters: slice thickness, 1.0 mm; increment, 1 mm; pitch, 1.078; a field of view, 15 cm; and a matrix of 512×512. The general characteristics and nodule radiographic characteristics of participants from the hospital information system were collected. General characteristics included age, gender, smoking behavior (smoking status, pack-years, and the number of years since quitting), and cancer history. Nodule radiographic characteristics comprised the maximum transverse size; location; and nodule type (nonsolid or ground-glass opacity, perifissural, part-solid, solid, and spiculation). The radiographic characteristics of PNs were obtained from the radiology report, documentation provided by an attending pulmonologist or thoracic surgeon, and by review of imaging by the research team. In the event of disagreement, the interpretation of the research team was used. Malignant or benign diagnosis of PNs was verified based on the pathologic examination of tissues obtained via surgery or biopsy. The surgical pathologic staging was determined based on the TNM guidelines classification criteria [17]. According to the World Health Organization classification to determine the histopathologic classification [18].

### Sample Collection and Storage

Plasma samples were collected from outpatients and inpatients of Henan Cancer Hospital, and the sample information was recorded in sample collection forms. Five milliliters of peripheral blood from the subject was drawn in a 5-ml K2EDTA anticoagulant tube (BD biosciences, Franklin Lakes, NJ, USA). The plasma sample's storage and transportation followed the instructions of the Nucleic Acid Extraction Reagent (Excellen Medical Technology Co., Ltd.).

### DNA Isolation and Bisulfite Conversion

Blood samples were collected before surgery, anesthesia, and adjuvant therapy. The collected specimens were processed within 4 h by centrifuging at 3000 g for 10 min at 4° C. Then, the collected plasma was transferred to a new tube and stored at −80° C. until use. DNA was extracted from plasma using the Nucleic Acid Extraction Reagent (Excellen Medical Technology Co., Ltd.) according to the instructions. In Brief, circulating DNA was extracted from 2 mL of plasma utilizing magnetic beads, then converted the unmethylated cytosine residue to uracil residue in DNA by a bisulfite reaction. After further purification, bisulfite-converted DNA (bisDNA) was eluted in 35 μL and ready for real-time PCR use.

### DNA Methylation Analysis

DNA methylation analysis was performed according to the diagnostic kit's instructions (Excellen Medical Technology Co., Ltd.). The eluted DNA was used as a template for fluorescent real-time PCR. Each PCR reaction mixture has a total reaction volume of 25 μL, including 12.5 μL reaction buffer, 2.5 μL primer mix, and 10 μL eluted DNA. Fluorescence PCR amplifications were performed on 96-well plates of Applied Biosystems 7500 Fast Real-Time PCR Systems. Each sample was carried out in triplicate. In addition to subject DNA samples, each plate also included positive controls (in vitro methylated leukocyte DNA), negative controls (normal leukocyte DNA or DNA from a known unmethylated cell line), and water blanks. The thermal profile for amplification reactions was 98° C. for 5 min, followed by 45 cycles at 95° C. for 10 s and 63° C. for 5 s to 58° C. for 30 s. In the PCR reaction, the primers and probes were designed to amplify the methylated sequences preferentially. During the PCR process, the methylated target sequence can be exclusively identified from unmethylated DNA. Increased inflorescent emission of the reporter dye can be detected on fluorescence channels of FAM, HEX, Texas Red, and CY5. The resulting data were analyzed by Applied Biosystems 7500 Fast Real-Time PCR System Sequence Detection Software v1.4.1.

A variety of primers and probes for PTGER4, RASSF1A, and SHOX2 genes (SEQ ID NOs:1-3) were designed, which were respectively equivalent to, complementary to, or hybridizable to at least 15 consecutive nucleotides of the sequences of PTGER4, RASSF1A, and SHOX2 genes or complementary sequences thereof and verified the effectiveness of the designed primers and probes with methylated and unmethylated nucleic acid sequences as templates. The following optimal primer sets and a primer set for the internal reference gene ACTB were selected based on real-time fluorescence PCR amplification results.

PTGER4 primer set 1:
primer 1:
(SEQ ID NO: 4)
5′-TTAGATATTTGGTGTTTTATCGATT-3′
primer 2:
(SEQ ID NO: 5)
5′-AAAAACTAAAACCCGCGTACAT-3′
blocking primer:
(SEQ ID NO: 6)
5′-TTTTATTGATTGGATTATTAATGTGATGGTGTATGTTG-3′P-3′
probe:
(SEQ ID NO: 7)
5′-JOE-ATAAACGACGTACGCCGTCACGTTAATA-BHQ1-3′
PTGER4 primer set 2
primer 1:
(SEQ ID NO: 8)
5′-TGGGTATTGTAGTCGCGAGTTATC-3′
primer 2:
(SEQ ID NO: 9)
5′-CTACGTAAACAAACGATTAACG-3′
blocking primer:
(SEQ ID NO: 10)
5′-TGTGAGTTATTGAGATTTATGTTGGGTAGTGT-C3-3′
probe:
(SEQ ID NO: 11)
5′-JOE-CAATCTATACGTCCAACGTACTCTTTTACGCGCTA-
BHQ1-3′
RASSF1A primer set 1
primer 1:
(SEQ ID NO: 12)
5′-GCGTTGAAGTCGGGGTTCG-3′
primer 2:
(SEQ ID NO: 13)
5′-CCGATTAAACCCGTACTTC-3′
blocking primer:
(SEQ ID NO: 14)
5′-TTGGGGTTTGTTTTGTGGTTTCGTTTGGTTTGT-C3-3′
probe:
(SEQ ID NO: 15)
5′-JOE-CGCTAACAAACGCGAACCGA-BHQ1-3′
RASSF1A primer set 2
primer 1:
(SEQ ID NO: 16)
5′-GGGAGTTTGAGTTTATTGA-3′
primer 2:
(SEQ ID NO: 17)
5′-GATACGCAACGCGTTAACACG-3′
blocking primer:
(SEQ ID NO: 18)
5′-CACATTAACACACTCCAACCAAATACAACCCTT-C3-3′
probe: SEQ ID NO: 19:
(SEQ ID NO: 19)
5′-JOE-CGCCCAACGAATACCAACTCC-BHQ1-3′
SHOX2 primer set 1
primer 1:
(SEQ ID NO: 20)
5′-GTTCGTGCGATTTCGGTC-3′
primer 2:
(SEQ ID NO: 21)
5′-TCGCTACCCCTAAACTCGA-3′
blocking primer:
(SEQ ID NO: 22)
5′-TGATTTTGGTTGGGTAGGTGGGATG-C3-3′
probe:
(SEQ ID NO: 23)
5′-FAM-CAACCAAATAATCTCCGTCCCGC-BHQ1-3′
SHOX2 primer set 2
primer 1:
(SEQ ID NO: 24)
5′-GGCGGGCGAAAGTAATC-3′
primer 2:
(SEQ ID NO: 25)
5′-CGAAAATCGCGAATATTCCG-3′
blocking primer:
(SEQ ID NO: 26)
5′-ACAAATATTCCACTTAAACCTATTAATCTCTATAAATTAAACA-
C3-3′
probe:
(SEQ ID NO: 27)
5′-FAM-AAAATCGAATCTACGTTTCCACGAAAA-BHQ1-3′
internal reference gene ACTB primers and probe
combination
primer 1:
(SEQ ID NO: 28)
5′-GTGATGGAGGAGGTTTAGTAAGT-3′
primer 2:
(SEQ ID NO: 29)
5′-CCAATAAAACCTACTCCTCCCTT-3′
probe:
(SEQ ID NO: 30)
5′-CY5-ACCACCACCCAACACACAATAACAAACACA-BHQ3-3′

All samples were within the range of the assay of sensitivity and reproducibility based on the amplification of internal reference standard [threshold cycle (Ct) value for β-Actin (ACTB)]. The 2^{-ΔCT} was calculated for each methylation detection replicate comparing it to the mean Ct for ACTB, the average value of triplicates of selected gene divided by the average value of triplicates of ACTB. For some samples replicates with the extremely low levels of DNA methylation in plasma, a Ct of 45 was used, creating a near-zero value for 2^{-ΔCT}.

### Statistical Analysis

Adapted from Han et al. [19], four well-established machine-learning algorithms were applied to predict a malignant or benign nodule (as a binary variable). The K-nearest neighbors (KNN), random forest (RF), support vector machine (SVM), and logistic regression (LR) algorithms used the DNA Methylation and clinically-relevant variables as candidate features. The performance of classifiers through fourfold cross-validation within the training set were evaluated. In detail, the training set was divided into four equal portions; then, during each of the four iterations, ¾ of the training data were applied first to train the classifiers (500 trees for RF, the radial kernel for SVM, other parameters set by default). Next, the trained classifiers were applied to the remaining ¼ of the training data for prediction. The predictions from all four iterations were combined and compared with the truth, then a receiver operator characteristic curve (ROC) was created and the area under the curve (AUC) was computed to evaluate the prediction capability for each model separately. Finally, the classifier trained from the whole training set was applied to an independent sample to independently validate the predictive power.

The variables for the final model of binomial logistic regression were selected through stepwise use of Akaike's information criterion (AIC). Then the selected variables were used to fit an ordinary logistic regression model and estimate the regression coefficients. The final constructed prediction model was validated in an independent sample for identifying malignant PNs.

The primary endpoint was the diagnostic accuracy for malignant PNs. Each model's diagnostic accuracy was assessed by calculating the area under the ROC curve (AUC) and 95% confidence intervals (CI). The non-parametric approach of DeLong et al. was used to compare the performance of the prediction model with that of the plasma biomarkers and the Mayo Clinic model [20]. The prediction model was developed in a cohort's training set and blindly validated in an additional set of subjects by comparing the calculated results with the final clinical diagnosis and the AUCs. A power analysis was conducted for the comparison between performance in the Mayo model versus the constructed prediction model with power (1−β) set at 0.8 and α=0.05. Based on published data [13], the expected AUC value of the Mayo model for identifying PNs was defined as 0.85. The analysis yielded a required sample size of 91 participants for detection 10% difference, estimated by the formula published previously [21]. R version 3.3.2 (The R Foundation for Statistical Computing) and MedCale Statistics were used for all analyses. P values <0.05 were considered to indicate statistical significance.

### Results

### Clinical Characteristics of Subjects

Altogether, 210 subjects were recruited, of which 120 were diagnosed with malignant PNs, 90 nodules were diagnosed with benign. The subjects were divided into a training cohort and a validation cohort by enrollment time. The initial series of 110 cases and controls were used for training and the subsequent series of 100 was used for validation. For each patient, only the largest nodule confirmed by histopathology was chosen for analysis. In the training cohort, 110 nodules, of which 63 were malignant, and in the validated cohort, 100 nodules, of which 57 were malignant (Table 1). Among persons with nodules, the rates of cancer in the two data sets were 57.3 and 57.0%, respectively. Subjects with lung cancer were generally older than subjects with benign nodules (58 vs 55 years). Of the subjects, 63 (57.3%) were male, and 69 (62.7%) were non-smokers. The 63 subjects with malignant PNs were diagnosed with adenocarcinomas (n=37), squamous cell carcinomas (n=14), small cell lung cancer (n=8) and, unclassified lung cancer patients (n=4). The LC patients consisted of 17 stage I, 21 stage II, and 25 stage III to IV cases. One hundred subjects with PNs were used as a validated cohort to confirm the prediction model for the differentiation of malignant from benign PNs. The cohort consisted of 57 subjects with malignant PNs (LC) and 43 subjects with benign PNs (Table 2). Of the patients with malignant PNs, 32 were diagnosed with adenocarcinomas, 14 were diagnosed with squamous cell carcinomas, 2 were diagnosed with small cell lung cancer, and 9 were unclassified lung cancer patients. The demographic and clinical parameters, including detailed information about the two cohorts' nodule characteristics, are shown in Tables 1 and 2, respectively.

TABLE 1
Subjects' Characteristics of Training Study

| | Subjects with | Subjects with |
|---|---|---|
| | Malignant PNs | Benign PNs |
| Characteristics | (n = 63) | (n = 47) |
| Clinical | | |
| Age (Years) | | |
| Median Age | 58 | 55 |
| Age Range | 36-77 | 26-70 |
| Sex | | |
| Male | 36 | 27 |
| Female | 27 | 20 |
| Smoking history | | |
| Non-smoker | 30 | 39 |
| Ex-smoker | 12 | 3 |
| Current smoker | 21 | 5 |
| Smoking pack-years | | |
| Mean Pack-years | 36.52 | 20 |
| (Smokers only) | | |
| Years quit (Smoker sonly) | 8.9 | 3 |
| Histology subtype | | |
| Adenocarcinoma | 37 | |
| Squamous cell carcinoma | 14 | |
| Small cell lung cancer | 8 | |
| Other | 4 | |
| Stage | | |
| I | 17 | |
| II | 21 | |
| III-IV | 25 | |
| Radiological | | |
| Nodule size (mm) | 21.46 (SD 10.52) | 11.89 (SD 6.81) |
| Nodule location | | |
| Left lower lobe | 9 | 6 |
| Left upper lobe | 17 | 12 |
| Right lower lobe | 15 | 15 |
| Right middle lobe | 3 | 4 |
| Right upper lobe | 19 | 10 |
| Nodule type (number) | | |
| Nonsolid or ground- | 17 | 13 |
| glass opacity | | |
| Perifissural | 6 | 5 |
| Part-solid | 9 | 8 |
| Solid | 13 | 11 |
| Spiculation | 18 | 10 |

Abbreviations:
PN, pulmonary nodule;
SD, standard deviation

TABLE 2
Subjects' Characteristics of Validation Study

| | Subjects with | Subjects with |
|---|---|---|
| | Malignant PNs | Benign PNs |
| Characteristics | (n = 57) | (n = 43) |
| Clinical | | |
| Age (Years) | | |
| Median Age | 62 | 54 |
| Age Range | 38-78 | 27-72 |
| Sex | | |
| Male | 39 | 29 |
| Female | 18 | 14 |
| Smoking history | | |
| Non-smoker | 25 | 30 |
| Ex-smoker | 12 | 4 |
| Current smoker | 20 | 9 |
| Smoking pack years | | |
| Mean Pack-years | 40.77 | 21.64 |
| (Smokers only) | | |
| Years quit (Smoker sonly) | 7.35 | 3.75 |
| Histology subtype | | |
| Adenocarcinoma | 32 | |
| Squamous cell carcinoma | 14 | |
| Small cell lung cancer | 2 | |
| Other | 9 | |
| Stage | | |
| I | 18 | |
| II | 20 | |
| III-IV | 19 | |
| Radiological | | |
| Nodule size (mm) | 21.83 (SD 10.88) | 11.22 (SD 7.56) |
| Nodule location | | |
| Left lower lobe | 12 | 10 |
| Left upper lobe | 12 | 10 |
| Right lower lobe | 11 | 11 |
| Right middle lobe | 5 | 4 |
| Right upper lobe | 17 | 8 |
| Nodule type (number) | | |
| Nonsolid or ground- | 17 | 13 |
| glass opacity | | |
| Perifissural | 8 | 6 |
| Part-solid | 9 | 7 |
| Solid | 13 | 10 |
| Spiculation | 10 | 7 |

Abbreviations:
PN, pulmonary nodule;
SD, standard deviation

### Diagnostic Accuracy of the Three Methylation Biomarkers for Identifying Malignant PNs

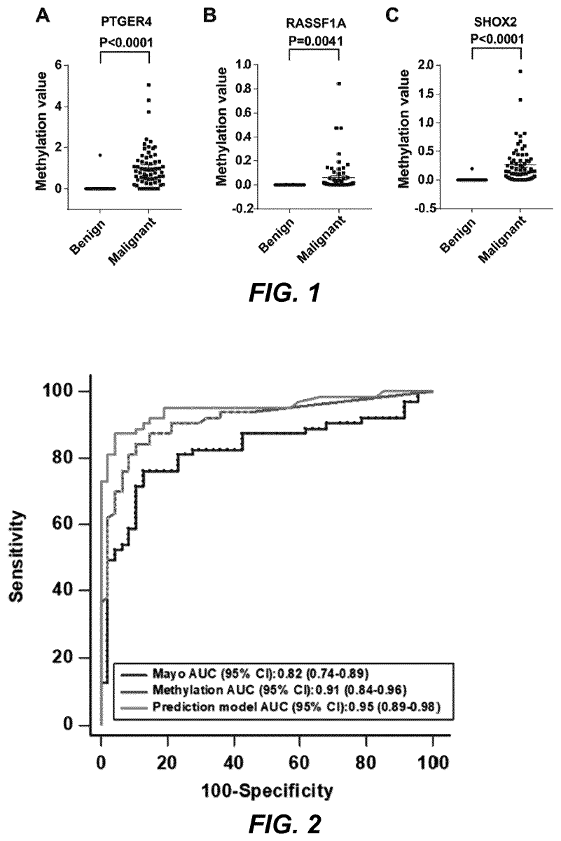

To determine the diagnostic values of the three methylation biomarkers, a quantitative analysis of promoter methylation in the plasma DNA samples from the training cohort of 110 subjects was performed using the diagnostic kit for the methylated gene of lung cancer (Excellen Medical Technology Co., Ltd.). Plasma expression level for each methylation biomarker was compared between two groups of subjects in the training set. As shown in FIG. 1, three methylation biomarkers displayed higher plasma expression levels in patients with malignant PNs compared to individuals with benign PNs (All P<0.01). The three methylation biomarkers present potential plasma biomarkers for identifying malignant PNs.

Receiver operating characteristic (ROC) curve analysis was further performed to evaluate the capability of using the three methylation biomarkers to discriminate patients with malignant PNs from patients having benign PNs. As shown in FIG. 2, the three DNA methylation used in combination yielded 0.912 AUC in identifying malignant from benign PNs. No statistically significant association was observed between the logistic model with subjects' age, gender, and smoking history (all p>0.05).

Developing a Prediction Model Based on the Methylation Biomarkers and Radiographic Features of PNs for Distinguishing Malignant from Benign PNs

Although use of the three DNA methylation showing promise with an AUC value of 0.912, it is not sufficient for identifying malignant PNs in the clinic. To improve the diagnostic accuracy for malignant PNs, a rigorous machine-learning approach was applied to assess the combined use of methylation biomarkers and all clinically-relevant variables in classifying PNs. First, within the training set, four well-established machine-learning algorithms were applied: K-nearest neighbors (KNN), random forest (RF), support vector machine (SVM), and logistic regression (LR) and evaluated their performance based on the area under the receiver operating characteristics curve (AUC) through fourfold cross-validation. It was found that the models of SVM and LR can accurately classify malignant from benign PNs with AUC of 0.92 and 0.93. Moreover, the best-performing algorithm, LR, achieved a high AUC of 0.96 on the independent test set (Table 3). These results indicated that the combined use of methylation biomarkers and clinically-relevant variables can effectively provide an independent approach to validate the classification of tumor subtypes.

TABLE 3
Accuracy and predictive value between four models

| Cross | | Sensi- | Speci- | | | Accu- | |
|---|---|---|---|---|---|---|---|
| Validation | Model | tivity | ficity | PPV | NPV | racy | AUC |
| 4-fold on | KNN | 0.83 | 0.86 | 0.90 | 0.80 | 0.83 | 0.84 |
| training | SVM | 0.89 | 0.85 | 0.89 | 0.86 | 0.87 | 0.92 |
| cohort | RF | 0.88 | 0.85 | 0.89 | 0.85 | 0.87 | 0.91 |
| | RL | 0.91 | 0.83 | 0.88 | 0.89 | 0.87 | 0.93 |
| Validated | KNN | 0.93 | 0.84 | 0.9 | 0.9 | 0.89 | 0.88 |
| in an | SVM | 0.93 | 0.93 | 0.91 | 0.91 | 0.93 | 0.96 |
| independent | RF | 0.91 | 0.93 | 0.89 | 0.89 | 0.92 | 0.95 |
| cohort | RL | 0.91 | 0.88 | 0.88 | 0.91 | 0.9 | 0.96 |

KNN K-nearest neighbors,
SVM support vector machine,
RF random forest,
RL logistic regression,
AUC area under the curve,
PPV positive predictive value,
NPV negative predictive value

The logistic regression model was used next through stepwise use of Akaike's information criterion (AIC) to select the variables for the final models. Once the AIC value no longer decreases, the stepwise regression analysis terminates and the optimal regression equation is output. Finally, the logistic regression model selected the methylation biomarkers (p<0.001) and diameter of PNs (p<0.001) as significant predictors for malignant PNs. Variables were presented in the prediction model by using the following formula: the probability of malignant PNs=e^{x}/(1+e^{x}), where e is the base of the natural logarithm and x=−4.433+1.066×(8.327−0.103*2^{-ΔCT.SHOX2}−0.184*2^{-ΔCT.RASSF1A}−0.077*2^{-ΔCT.PTGER4)}+0.151×Diameter of PNs (the detection results of the methylation levels were obtained with primer set 1 for each of the biomarker genes). Then, the performance of this prediction model in the training set was evaluated, which produced 0.951 AUC in identifying malignant from benign PNs (FIG. 2). It has been reported that several prediction models based on PNs parameters on CT images and clinical characteristics of subjects developing to predict the probability of malignant PNs [13,14,15,16], of which the Mayo Clinic model is a commonly used one. Also applied was the equation of the Mayo Clinic model: Probability of Malignancy=e^{x}/(1+e^{x}), x=−6.8272+(0.0391×Age)+(0.7917×Smoking history)+(1.3388×Cancer)+(0.1274×Diameter)+(1.0407×Spiculation)+(0.7838×Upper) [13] to predict malignant PNs in the training cohort of 110 subjects, as shown in FIG. 2. The AUC value obtained by the Mayo Clinic model was 0.823, and the value was similar to the previous reports [13,14,15]. The AUC value of the prediction model (0.951, 95% CI:0.892-0.983) was significantly higher than the panel of the three methylation biomarkers (0.912, 95% CI: 0.843-0.958, p=0.013) used alone and the Mayo Clinic model (0.823, 95% CI:0.739-0.890, p=0.001).

### Validating the Prediction Model for Identifying Malignant PNs in an Independent Cohort

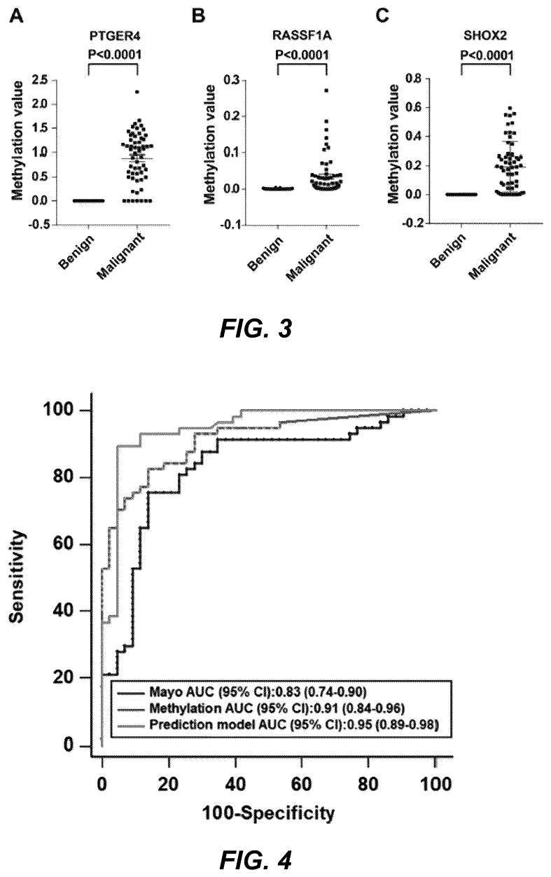

Firstly, the expression of the DNA methylation panel in an independent cohort was confirmed. The three-gene methylation biomarkers displayed higher plasma expression levels in patients with malignant PNs compared to individuals having benign PNs (All p<0.0001) (FIG. 3). The observations were in agreement with the findings observed in the above training test, which indicated that the gene methylation could be reproducibly measured. Then, the diagnostic performance of the three models was evaluated. The AUC value of the prediction model in the validated cohort (0.948) was similar to in the training cohort (0.951). As shown in FIG. 4, the AUC value of the prediction model was significantly higher than the panel of the biomarkers (0.912, 95% CI: 0.84-0.96) and the Mayo Clinic model (0.829, 95% CI: 0.94-0.90). The optimal cut-offs obtained in the training set were used to determine the prediction model's diagnostic performance in the validated cohort. The prediction model produced a sensitivity of 89.5% and a specificity of 95.4%. Taken together, these results confirmed that the prediction model had the potential for estimating malignant PNs among individuals with CT-detected PNs.

### Discussion

Low-dose spiral computed tomography (LDCT), a reliable screening tool for early detection of lung cancer, was severely limited by its low specificity [4, 5]. LDCT dramatically increases the number of indeterminate pulmonary nodules (PNs), whereas most PNs are ultimately false positives [22]. It is clinically significant to develop new methods that can precisely identify malignant from benign PNs safely and cost-effectively.

Some clinical/radiological characteristics-based models have shown the potential to identify malignant PNs [13,14,15]. The finding from the present study confirmed the previous observations. However, the moderate sensitivity and specificity of these models limit the application in clinical. DNA methylation plays a vital role in tumorigenesis at an early stage [23,24,25]. That makes DNA methylation alterations among the most promising candidates in biomarker research. To improve the diagnostic accuracy of lung cancer, various DNA methylation biomarkers have been explored. Among them, PTGER4, RASSF1A, and SHOX2 methylation biomarkers showed high potential in the diagnosis and prognosis of lung cancer. Kneip C et al. performed DNA methylation analysis of the SHOX2 gene in blood, the result showed a sensitivity of 60% and specificity of 90% in the diagnosis of lung cancer [11]. Hu et al. reported that promoter hypermethylation of RASSF1A occurs frequently in lung cancer and is frequently found in small cell lung cancer [12]. Besides, Weiss G et al. validated that SHOX2/PTGER4 DNA methylation marker panel could discriminate between patients with malignant and nonmalignant lung disease with an AUC value of 0.88 [9]. Inspired by these studies, detection of PTGER4, RASSF1A, and SHOX2 methylation biomarkers were combined for estimating malignant from benign PNs in a training cohort. The three methylation biomarkers used in combination produced an AUC value of 0.912. Despite showing promise, the diagnostic accuracy also needed to be further improved. A novel lung nodule risk prediction model was developed by integrating the three DNA methylation biomarkers with one radiological variable of PNs to estimate the probability of malignancy in PNs. The prediction model has a higher AUC value than the Mayo Clinic model or the panel of biomarkers used alone. Furthermore, in an independent cohort, the prediction model's performance validated, further confirming the tremendous potential for detecting malignant PNs. The current findings suggested that the prediction model with three DNA methylation biomarkers and the diameter of PNs may potentially guide the management of CT screening results.

Based on the Food and Drug Administration criteria, a disease with a 5% prevalence, the screening test should have a sensitivity exceeding 95% when the specificity <95%, and vice versa [26]. The prevalence of lung cancer in high-risk populations is 1 to 3%, while LDCT has about 90% sensitivity and only 61% specificity, which is prone to produce a high false-positive rate. The ideal prediction model should have >95% specificity and appropriate sensitivity for identifying malignant PNs, thus, could augment the performance of LDCT for lung cancer screening [27]. The result appears promising; the developed prediction model achieved a sensitivity of 87.3% and a specificity of 95.7% with an AUC value of 0.951 in malignant PNs diagnosis, which suggested that the prediction model does possess the required diagnostic performance for routine clinical application.

However, the study also has some limitations. The sample size is small. The exact number of subjects in some histological subtype groups, such as small cell lung cancer, may be insufficient. A large sample size is needed in further studies to confirm the results. Furthermore, subjects in this study were recruited from hospital-based patients with PNs. The subjects might not be representative of a population-based LDCT screening setting for lung cancer. A large trial of population-based LDCT screening will be conducted to confirm the prediction model's performance in identifying malignant PNs.

### CONCLUSIONS

In summary, a simple prediction model based on DNA methylation biomarkers with radiological characteristics that could identify malignant from benign nodules detected by LDCT was developed. Future use of the prediction model could reduce costs and avoid invasive diagnostic procedures for patients with benign PNs, at the same time, allowing immediate treatment for lung cancer patients. This prediction model could be used in combination with LDCT to improve the over-all diagnosis of lung cancer. Nevertheless, undertaking a prospective study of the prediction model for malignant PNs in an extensive population-based LDCT screening is required.

Listed below are some nucleic acid sequences mentioned herein.

human PTGER4 gene
SEQ ID NO: 1
CTTCTTCAGCCTGTCCGGCCTCAGCATCATCTGCGCCATGAGTGTCG
AGCGCTACCTGGCCATCAACCATGCCTATTTCTACAGCCACTACGTGGACAAGCGAT
TGGCGGGCCTCACGCTCTTTGCAGTCTATGCGTCCAACGTGCTCTTTTGCGCGCTGCC
CAACATGGGTCTCGGTAGCTCGCGGCTGCAGTACCCAGACACCTGGTGCTTCATCGA
CTGGACCACCAACGTGACGGCGCACGCCGCCTACTCCTACATGTACGCGGGCTTCAG
CTCCTTCCTCATTCTCGCCACCGTCCTCTGCAACGTGCTTGTGTGCGGCGCGCTGCTC
CGCATGCACCGCCAGTTCATGCGCCGCACCTCGCTGGGCACCGAGCAGCACCACGC
GGCCGCGGCCGCCTCGGTTGCCTCCCGGGGCCACCCCGCTGCCTCCCCAGCCTTGCC
GCG
human RASSF1A gene
SEQ ID NO: 2
CTGCGAGAGCGCGCCCAGCCCCGCCTTCGGGCCCCACAGTCCCTGCA
CCCAGGTTTCCATTGCGCGGCTCTCCTCAGCTCCTTCCCGCCGCCCAGTCTGGATCCT
GGGGGAGGCGCTGAAGTCGGGGCCCGCCCTGTGGCCCCGCCCGGCCCGCGCTTGCT
AGCGCCCAAAGCCAGCGAAGCACGGGCCCAACCGGGCCATGTCGGGGGAGCCTGA
GCTCATTGAGCTGCGGGAGCTGGCACCCGCTGGGCGCGCTGGGAAGGGCCGCACCC
GGCTGGAGCGTGCCAACGCGCTGCGCATCGCGCGGGGCACCGCGTGCAACCCCACA
CGGCAGCTGGTCCCTGGCCGTGGCCACCGCTTCCAGCCCGCGGGGCCCGCCACGCA
CACGTGGTGCGACCTCTGTGGCGACTTCATCTGGGGCGTCGTGCGCAAAGGCCTGCA
GT
human SHOX2 gene
SEQ ID NO: 3
TGGCTCTCTGCCTACCGCAAACTTGCTGGTCTAATTTAGGAACAATT
GGGCCGAAAGGTATCAGCGAGAGCAACAGACCCCGGTGTTGTGCCGCACAGGGAGC
CGCATCCGCAGACGCCCCTCGCTGCCCCTGGGCTCGGGCCAAACCCTGCATAAGGTC
CCCTGGACAGCCAGGTAATCTCCGTCCCGCCTGCCCGACCGGGGTCGCACGAGCAC
AGGCGCCCACGCCATGTTGGCTGCCCAAAGGGCTCGCCGCCCAAGCCGGGCCAGAA
GGCAGGAGGCGGAAAACCAGCCTCCGGTGGCGGGCGAAAGCAACCGCTCTTTCTGT
TCTCTCTTCGCCCTCCCTCGTGGAAACGCAGACTCGACCCTAAACGCTTAACCCACA
GAGATCAACAGGTTCAAGCGGAATATTCGCGATCCTCGGTTTCTATTGGTTGCTCAA
AGCCTTTTCATGCAACCAGCAGCTCGGATGTTTAATAAAATATGAATT

### REFERENCES

1. Siegel R L, Miller K D, Jemal A. Cancer statistics, 2020. CA Cancer J Clin. 2020; 70(1):7-30.
2. Begum S, Brait M, Dasgupta S, et al. An epigenetic marker panel for detection of lung cancer using cell-free serum DNA. Clin Cancer Res. 2011; 17(13):4494-503.
3. Blandin Knight S, Crosbie P A, Balata H, Chudziak J, Hussell T, Dive C. Progress and prospects of early detection in lung cancer. Open Biol. 2017; 7(9):170070.
4. Patz E F Jr, Pinsky P, Gatsonis C, et al. Overdiagnosis in low-dose computed tomography screening for lung cancer. JAMA Intern Med. 2014; 174:269-74.
5. National Lung Screening Trial Research Team, Aberle D R, Adams A M, et al. Reduced lung-cancer mortality with low-dose computed tomographic screening. N Engl J Med. 2011; 365(5):395-409.
6. Diederich S, Das M. Solitary pulmonary nodule: detection and management. Cancer Imaging. 2006; 6(Spec No A): S42-S46. Published 2006 Oct. 31.
7. National Lung Screening Trial Research Team, Aberle D R, Berg C D, et al. The National Lung Screening Trial: overview and study design. Radiology. 2011; 258(1):243-53.
8. Hulbert A, Jusue-Torres I, Stark A, et al. Early detection of lung Cancer using DNA promoter Hypermethylation in plasma and sputum. Clin Cancer Res. 2017; 23(8):1998-2005.
9. Weiss G, Schlegel A, Kottwitz D, König T, Tetzner R. Validation of the SHOX2/PTGER4 DNA methylation marker panel for plasma-based discrimination between patients with malignant and nonmalignant lung disease. J Thorac Oncol. 2017; 12(1):77-84.
10. Zhang C, Yu W, Wang L, et al. DNA Methylation Analysis of the SHOX2 and RASSF1A Panel in Bronchoalveolar Lavage Fluid for Lung Cancer Diagnosis. J Cancer. 2017; 8(17):3585-91. Published 2017 Sep. 30.
11. Kneip C, Schmidt B, Seegebarth A, et al. SHOX2 DNA methylation is a biomarker for the diagnosis of lung cancer in plasma. J Thorac Oncol. 2011; 6(10):1632-8.
12. Hu H, Zhou Y, Zhang M, Ding R. Prognostic value of RASSF1A methylation status in non-small cell lung cancer (NSCLC) patients: a meta-analysis of prospective studies. Biomarkers. 2019; 24(3):207-16.
13. Swensen S J, Silverstein M D, Ilstrup D M, Schleck C D, Edell E S. The probability of malignancy in solitary pulmonary nodules. Application to small radiologically indeterminate nodules. Arch Intern Med. 1997; 157(8):849-55.
14. Gould M K, Ananth L, Barnett P G, Veterans Affairs SNAP Cooperative Study Group. A clinical model to estimate the pretest probability of lung cancer in patients with solitary pulmonary nodules. Chest. 2007; 131(2):383-8.
15. Schultz E M, Sanders G D, Trotter P R, et al. Validation of two models to estimate the probability of malignancy in patients with solitary pulmonary nodules. Thorax. 2008; 63(4):335-41.
16. McWilliams A, Tammemagi M C, Mayo J R, et al. Probability of cancer in pulmonary nodules detected on first screening CT. N Engl J Med. 2013; 369(10):910-9.
17. Ettinger D S, Wood D E, Akerley W, et al. Non-small cell lung cancer, version 1.2015. J Natl Compr Cancer Netw. 2014; 12(12):1738-61.
18. Travis W D, Brambilla E, Nicholson A G, et al. The 2015 World Health Organization Classification of lung tumors: impact of genetic, clinical and radiologic advances since the 2004 classification. J Thorac Oncol. 2015; 10(9):1243-60.
19. Han L, Yuan Y, Zheng S, Yang Y, Li J, Edgerton M E, Diao L, Xu Y, Verhaak R G W, Liang H. The pan-Cancer analysis of pseudogene expression reveals biologically and clinically relevant tumor subtypes. Nat Commun. 2014; 5:3963.
20. Cui X, Heuvelmans M A, Han D, et al. Comparison of Veterans Affairs, Mayo, Brock classification models and radiologist diagnosis for classifying the malignancy of pulmonary nodules in Chinese clinical population. Transl Lung Cancer Res. 2019; 8(5):605-13.
21. Hajian-Tilaki K. Sample size estimation in diagnostic test studies of biomedical informatics. J Biomed Inform. 2014; 48:193-204.
22. Blanchon T, Brechot J M, Grenier P A, et al. Baseline results of the Depiscan study: a French randomized pilot trial of lung cancer screening comparing low dose CT scan (LDCT) and chest X-ray (CXR). Lung Cancer. 2007; 58(1):50-8.
23. Locke W J, Guanzon D, Ma C, et al. DNA Methylation Cancer Biomarkers: Translation to the Clinic. Front Genet. 2019; 10:1150. Published 2019 Nov. 14.
24. Fukushige S, Horii A. DNA methylation in cancer: a gene silencing mechanism and the clinical potential of its biomarkers. Tohoku J Exp Med. 2013; 229(3):173-85.
25. Klutstein M, Nejman D, Greenfield R, Cedar H. DNA methylation in Cancer and aging. Cancer Res. 2016; 76(12):3446-50.
26. Ma J, Guarnera M A, Zhou W, Fang H, Jiang F. A prediction model based on biomarkers and clinical characteristics for detection of lung Cancer in pulmonary nodules. Transl Oncol. 2017; 10(1):40-5.
27. Lin Y, Leng Q, Jiang Z, et al. A classifier integrating plasma biomarkers and radiological characteristics for distinguishing malignant from benign pulmonary nodules. Int J Cancer. 2017; 141(6):1240-8.

1. A method of predicting lung cancer in a subject, the method comprising:
using multiple methylation biomarker genes in combination with a radiological characteristic of a pulmonary nodule for the prediction of lung cancer in the subject.

2. The method of claim 1, wherein the multiple methylation biomarker genes are selected from PTGER4, RASSF1A and SHOX2.

3. The method of claim 1, wherein the multiple methylation biomarker genes are PTGER4, RASSF1A and SHOX2.

4. The method of claim 1, wherein the radiological characteristic is a size of the pulmonary nodule.

5-9. (canceled)

10. A method for identifying lung cancer in a subject with a pulmonary nodule, comprising:
determining a radiological characteristic of the pulmonary nodule the subject;
detecting methylation levels of multiple methylation biomarker genes in a sample from the subject; and
assessing whether the subject has a lung cancer or not by using the radiological characteristic in combination with the detected methylation levels.

11. (canceled)

12. The method of claim 10, wherein the multiple methylation biomarker genes are PTGER4, RASSF1A and SHOX2.

13. The method of claim 12, wherein the detection of methylation levels is performed by using a methylation-specific primer pair.

14. The method of claim 13, wherein the methylation-specific primer pair for PTGER4 gene comprises the sequences of SEQ ID NOs:4 and 5 or the sequences of SEQ ID NOs:8 and 9; the methylation-specific primer pair for RASSF1A gene comprises the sequences of SEQ ID NOs:12 and 13 or the sequences of SEQ ID NOs:16 and 17; and the methylation-specific primer pair for SHOX2 gene comprises the sequences of SEQ ID NOs:20 and 21 or the sequences of SEQ ID NOs:24 and 25.

15. The method of claim 10, wherein the radiological characteristic is a size of the pulmonary nodule.

16. The method of claim 15, wherein the radiological characteristic is a diameter of the pulmonary nodule.

17. The method of claim 16, wherein the subject has multiple pulmonary nodules, and the radiological characteristic is the diameter of a largest pulmonary nodule.

18. The method of claim 16, wherein the diameter is a CT-derived diameter.

19. The method of claim 16 wherein the diameter is a LDCT-derived diameter.

20. The method of claim 10, wherein the assessing is based on a prediction model constructed by using a training cohort consisting of patients with a malignant pulmonary nodule and patients with a benign pulmonary nodule, and wherein information about the pulmonary nodule radiological characteristic and levels of methylation biomarker genes as well as whether the subject is a lung cancer patient is known in the training cohort.

21. The method of claim 20, wherein the prediction model is a logistic regression model.

22. The method of claim 20, wherein the lung cancer is a malignant pulmonary nodule.

23. The method of claim 22, wherein the prediction model is presented as:
a probability of malignant PN=e^{x}/(1+e^{x}), where e is the base of a natural logarithm and x=−4.433+1.066×(8.327−0.103*2^{-ΔC}T.SHOX2−0.184*2^{-ΔCT.RASSF1A}−0.077*2^{-ΔCT.PTGER4)}+0.151×the diameter of the pulmonary nodule.

24. A kit for identifying lung cancer in a subject with a pulmonary nodule, comprising agents for detecting methylation levels of multiple methylation biomarker genes PTGER4, RASSF1A and SHOX2 in a sample from the subject.

25. The kit of claim 24, wherein the agents for detecting methylation levels comprise methylation-specific primer pairs, and wherein the methylation-specific primer pair for PTGER4 gene comprises the sequences of SEQ ID NOs:4 and 5 or the sequences of SEQ ID NOs:8 and 9; the methylation-specific primer pair for RASSF1A gene comprises the sequences of SEQ ID NOs:12 and 13 or the sequences of SEQ ID NOs:16 and 17; and the methylation-specific primer pair for SHOX2 gene comprises the sequences of SEQ ID NOs:20 and 21 or the sequences of SEQ ID NOs:24 and 25.

26. The kit of claim of 24, wherein the kit further comprises an instruction indicating that the methylation levels are used in combination with a radiological characteristic of the pulmonary nodule to identify whether the subject is a lung cancer patient, wherein the radiological characteristic is the diameter of the pulmonary nodule.

27. (canceled)

28. (canceled)
